(19) United States
(12) Patent Application Publication
(10) Pub. No.: US 2006/0129078 A1
(43) Pub. Date: Jun. 15, 2006

(54) Balanced tension arm and shoulder splint

(76) Inventor: Walter J. Ledergerber, Dublin, CA (US)

Correspondence Address: O'MELVENY & MYERS LLP, 610 NEWPORT CENTER DRIVE, 17TH FLOOR, NEWPORT BEACH, CA, 92660, US

(21) Appl. No.: 11/290,629

(22) Filed: Nov. 29, 2005

Related U.S. Application Data
(60) Provisional application No. 60/667,520, filed on Apr. 1, 2005.
(60) Provisional application No. 60/683,064, filed on May 20, 2005.

(51) Int. Cl. A61F 5/00

(52) U.S. Cl. 602/20

(57) ABSTRACT

A balanced tension splint for the arm is disclosed. The device applies constant pressure to the shoulder in a manner that helps prevent its becoming dislocated as it serves to support the weight of the arm. The splint is designed to support the dysfunctional/injured arm and shoulder by applying the weight of the opposite arm in dynamic counter-tension. In addition, the splint is designed to apply pressure onto the shoulder to keep the head of the humerus engaged with the glenoid fossa and coracoid process. Balanced dynamic tension and support is accomplished by the linkage of two tension systems via a comfortable U-shaped collar. The splint is used for the effective management of arm and shoulder problems in strokes or trauma to the upper extremity.

### RELATED APPLICATIONS

This application claims the benefit of provisional U.S. patent application Ser. No. 60/667,520, filed Apr. 1, 2005, and provisional U.S. Patent application Ser. No. 60/683,064, filed May 20, 2005, both entitled “BALANCED—TENSION ARM AND SHOULDER SPLINT”, and both incorporated herein by reference as if fully set forth herein.

### FIELD OF THE INVENTION

This invention relates to assistive devices in the acute and chronic medical care of the upper extremity and shoulder in strokes and trauma.

### BACKGROUND OF THE INVENTION

In complex clinical situations in which the upper extremity is severely injured and where rest is advised, or where the arm may be grossly decreased in function as for example in stroke, the weight of the arm over an extended period of time can result in dislocation of the shoulder. This event may be unrecognized with the use of conventional arm splints used in such a clinical setting. The result is that severe abnormal stretching of the ligaments of the joint cause pain and secondary injury to joint cartilages, ligaments and tendons which serve to cause the patient to avoid movement and exercise of the arm. Impingement of nerves serving motor and sensory functions including touch and proprioception may result. This inevitably leads to a complicating disability over and above the initial injury.

Wound healing and recovery of function often requires rehabilitative physical therapy administered by treatment specialists over an extended period of time. The proper positioning and support of the arm during periods of rest is of paramount importance in preventing the complication of shoulder dislocation known commonly as a “frozen” shoulder.

Conventional splints commonly apply unusual pressure onto the patient's neck and are difficult to fit properly. It would be highly desirable to have available in the clinical setting a comfortable and effective assistive device to enhance the treatment and rehabilitation of the arm and shoulder.

### SUMMARY OF THE INVENTION

The present invention serves to support the arm and shoulder simultaneously by applying tension between an arm supporting member A and a curved line of fixation F on a unique collar C of the device. The tension is preferably applied via a stretchy material B which simultaneously applies downward and inward pressure on the shoulder to counteract a potential subluxation of the shoulder while also supporting the weight of the extremity.

The entire system of arm support and shoulder reduction applied to the first arm is supported by the counterbalancing weight of the second arm. The arms, thusly, are in a balanced relationship of dynamic counter-tension maintained by means of the open collar C which rests comfortably around the neck. The neck experiences no laterally-directed pressure, yet the entire weight of the affected arm is supported by the invented device by directing traction forces around the neck via the unique collar.

Fitting and adjusting the device is preferably accomplished by selecting an appropriately sized sheet member B, or by selecting a special sheet member B of calibrated tension, and attaching it to canvas member CZ of the collar C by means of the zipper Z or other means. Tension is adjustable also by selecting appropriate shoulder insert pads P. Anterolateral and/or posterolateral positioning adjustments can be made if needed by adjusting tension in means X and Y via suspension adjustment means SAM such as a slidable fixture or variable length adjustment device.. Balancing arm position and tension can furthermore be made by supporting the position of the arm in the sling on the affected side by inflating a unique soft adjustable arm support H or a wedge of closed cell foam. Cushioning M may also be used liberally by affixing felt, sheepskin, air bladder or foam cushion to the collar as indicated, by using Velcro® or other adhesive. The selection of materials for the construction of the invented device herein will be apparent to those experienced in the art.

### BRIEF DESCRIPTION OF THE DRAWINGS

The above objects and advantages of this invention will be more easily understood with reference to the following drawings and detailed descriptions. In the drawings:

FIG. 1 is a plan view of the invented splint. It is comprised of two symmetrical hemi-splints each joined to the collar and thereby to each other.

FIG. 2 shows detail of the collar and symmetrical attachment mechanism thereto.

FIG. 3 shows a side view of the splint in position of use supporting an arm.

FIG. 4 shows a posterior view of the splint on a wearer in a position of function.

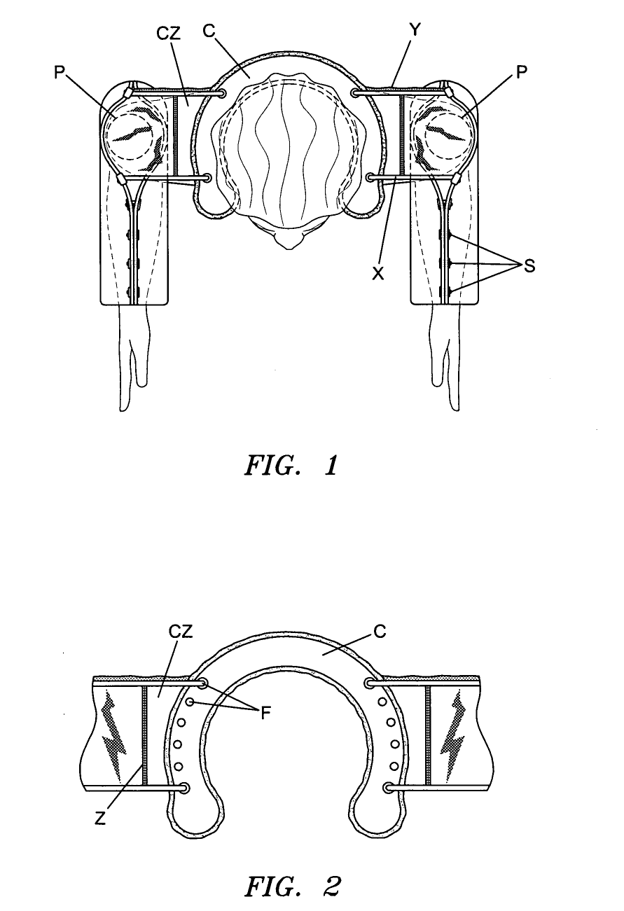

### DETAILED DESCRIPTION OF THE INVENTION

The splint herein may be manufactured from materials and by methods typically used in the field of medical assistive devices including moldings, sheet materials, adhesive structural means, customized rigging and so forth. The collar may be made of solid plastic or plastic blow molding or solid or die formed sheet metal or die formed and spot welded metal rod element. The mechanism of attachment of woven material to collar may comprise riveting, gluing, co-molding, wrapping or other suitable techniques known to those skilled in the art. The method of attachment of densely woven materials or stretchy materials such as Lycra® Spandex to each other may comprise sewing or the use of zippers or by using hooks or lashing methods or Velcro®, etc. Any and all methods and means are selected herein as preferred embodiments or preferred methods of flexible attachment, although zippers are best because they offer security, flexibility, wash ability and serve to provide for problem-free attachment and detachment.

The stretch material may be comprised of a dense nylon woven mesh such as Lycra® Spandex preferably attached to the collar by the aforementioned zipper and likewise may be joined to the arm support component by zipper or simply by sewing. The forearm support component or sleeve is of common construction, typically a canvas sheet attachable to itself marginally at a line parallel to the forearm and perpendicular to the upper arm.

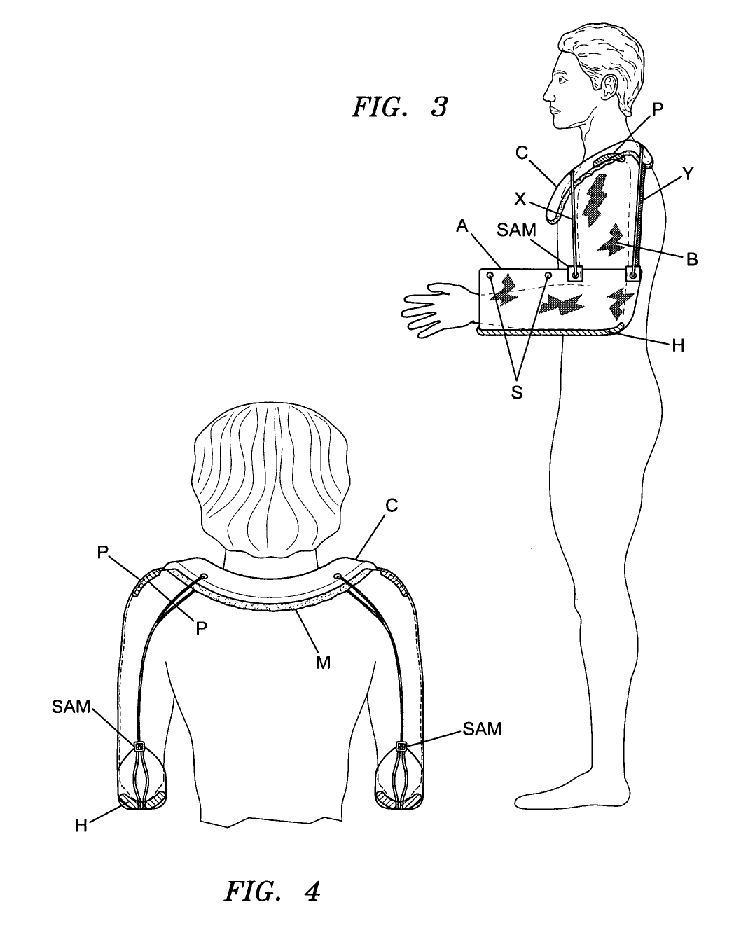

The unaffected arm serves as a counter weight for the affected arm by virtue of the symmetrical attachment of the hemi-splints to the collar which serves as a bridge around the neck. The collar itself is multiply curved so that it is positioned comfortably around the base of the neck and is cushioned by sheepskin, foams, air bladders and the like, and sized to accommodate the wearer's anatomy. The stretchy material component B, acting in concert with the tensioning means X and Y, exerts downward and inward pressure onto the shoulder and thereby onto the head of the humerus while simultaneously supporting the arm support component A. Adjustment of pressure onto the shoulder is accomplished principally by selecting the appropriately calibrated stretch material B for assembly into the final configuration. Additional adjustment of the tension applied to the shoulder may be accomplished by inserting an appropriately thick pad P into a special pocket sewn to the undersurface of the stretch material B. Alternatively, an air-fillable bladder may serve in place of pad P to adjust pressure on the shoulder joint. Furthermore, adjustment of tension may be accomplished by tightening or loosening means X and/or Y. Additional tension adjustment options are available via the attachment points S for means X and Y. Cushioning means H inserted underneath the affected arm—configured in FIG. 3 as an adjustable air bladder also serves as yet another mechanism to calibrate the tensioning system. Adjunctive devices facilitating treatment, communication, entertainment and so forth are integrated into the collar or soft material portions of the splint.

Though the invention has been described with respect to specific embodiments, many variations and modifications will become apparent to those skilled in the art. It is Therefore the intention and expectation that the appended claims be interpreted as broadly as possible in view of the prior art to include all such variations and modifications.

I claim:

1. A splint for use with a mammal having arms and a neck, comprising:
first and second symmetrical hemi-splints,
a U-shaped floating collar, the collar being adapted for disposal around the neck, and
connectors coupling the first and second hemi-splints through the floating collar whereby a balanced-tension arm and shoulder splint is formed.

2. The splint in claim 1, where tension on the affected arm side equals the tension on the opposite side.

3. The splint in claim 1, where the tension is directed proximally from the elbows toward the collar.

4. The splint in claim 1, where pressure is applied external to the deltoid muscle and directed inward toward the shoulder joint by a stretch material element of the splint, while also supporting the weight of the arm.

5. The splint in claim 1, where the adjustment of tension on the affected side is accomplished by affixation of the stretch material component of the splint to one of at least two lines of attachment on the collar component of the splint.

6. The splint in claim 1, where tension is adjusted by exchanging the stretchy sheet component on either one or both sides for a different sheet with calibrated tension.

7. The splint in claim 1, where the stretchy sheet component on the unaffected side is replaced by a sheet with no stretch.

8. The splint in claim 1, where the stretchy sheet component on both sides is exchanged for a sheet without any stretch.

9. The splint in claim 1, where the adjustment of tension on either or both sides is further accomplished via the use of soft spacer pads placed beneath the stretchy sheet component overlying the deltoid muscle.

10. The splint in claim 9, where the function of the spacer pad is replaced by an inflatable bladder.

11. The splint in claim 1, where the adjusting of tension and/or arm position is accomplished by separate or integrated tensioning means, where the medial ends of said tensioning means are attached to the collar, and where there are multiple optional points of attachment of the distal end of the tensioning means to the arm component at S.

12. The splint in claim 1, where calibration of the ideal tension is also accomplished via the inflatable arm support cushioning means by adjusting air volume in the air bladder H.

13. The splint in claim 1, where a heating element and its temperature control component are integrated into the construction of the collar or splint.

14. The splint in claim 1, where a neuromuscular stimulation or monitoring device and controls are integrated into the construction of the collar or splint.

15. The splint in claim 14, where the heating element and controls are replaced by an entertainment or communication device.
